(19) United States
(12) Patent Application Publication
(10) Pub. No.: US 2022/0096159 A1
(43) Pub. Date: Mar. 31, 2022

(54) SYSTEMS AND METHODS FOR MODELLING A HUMAN SUBJECT

(71) Applicant: KONINKLIJKE PHILIPS N.V., Eindhoven (NL)

(72) Inventors: Cornelis Petrus Hendriks, Eindhoven (NL); Lieke Gertruda Elisabeth Cox, Eindhoven (NL); Valentina Lavezzo, Eindhoven (NL); Murtaza Bulut, Eindhoven (NL); Lucas Johannes Anna Maria Beckers, Eindhoven (NL)

(21) Appl. No.: 17/351,420

(22) Filed: Jun. 18, 2021

Related U.S. Application Data
(60) Provisional application No. 63/083,186, filed on Sep. 25, 2020.

(51) Int. Cl. A61B 34/10; A61B 90/00

(52) U.S. Cl. CPC A61B 34/10; A61B 90/37; A61B 2090/378; A61B 2090/374; A61B 2034/105

(57) ABSTRACT

It is proposed to combine a multi-organ in-vitro system with a multi-organ in-silico system to provide improved modelling/prediction of multi-organ interaction(s) for a subject. By way of example, a subject-specific multi-organ in-vitro system may undergo perturbation. The response of the other organ(s) to the perturbation modelled by the in-vitro model may then be identified, and this response may then be used in the in-silico model to predict the outcome(s) of the perturbation(s).

### FIELD OF THE INVENTION

The invention relates to modelling human subjects, and more particularly to the field of subject-specific models of biological function (commonly referred to as digital twins).

### BACKGROUND OF THE INVENTION

A recent development in healthcare is the so-called ‘digital twin’ concept. In this concept, a digital representation or computational simulation (i.e. the digital twin) of a physical system is provided and connected to its physical counterpart, for example through the Internet of things as explained in US 2017/286572 A1 for example. Through this connection, the digital twin typically receives data pertaining to the state of the physical system, such as sensor readings or the like, based on which the digital twin can predict the actual or future status of the physical system, e.g. through simulation.

Such digital twin technology is also becoming of interest in the medical field, as it provides an approach to more efficient medical care provision. For example, a digital twin may be built using imaging data of a subject (i.e. patient), e.g. a person suffering from a diagnosed medical condition as captured in the imaging data.

A digital twin of a subject (i.e. a subject-specific computational simulation) may serve a number of purposes. Firstly, the digital twin (rather than the patient) may be subjected to a number of virtual tests, e.g. treatment plans, to determine which treatment plan is most likely to be successful to the patient. This reduces the number of tests that physically need to be performed on the actual patient. Secondly, the digital twin of a subject may be used to predict the onset, treatment (outcome) or development of medical conditions of the subject.

However, in a real human, multiple organs work together in biological systems (respiratory, cardiovascular, digestive system etc.). Communication and interaction takes place via physical, neurological, endocrine, biochemical and immunological pathways. Similarly, in a digital twin of a subject, organs of different type and nature work together in sub-systems (organ systems) and a super-system (human body).

A problem in the computational simulation of human biological systems is the characterization and quantification of the interactions between organs. The interactions can be difficult or impossible to measure with external measurement devices or tests (e.g. sensors, wearable monitors, imaging investigations, blood tests, etc.). If there is no data available about the interactions, the computational models may be inaccurate and thus be of limited use.

### SUMMARY OF THE INVENTION

The invention is defined by the claims.

According to examples in accordance with an aspect of the invention, there is provided a system for subject-specific clinical decision support comprising:

an in-vitro model configured to model a plurality of organs of a subject;

a processor arrangement adapted to implement an in-silico digital twin model configured to model the plurality of organs of the subject; and

an input interface configured to provide input data to the in-vitro model, the input data being representative of a change to the subject,

wherein the in-vitro model is configured to evaluate the input data received from the input interface to generate an in-vitro response;

and wherein the processor arrangement is configured to:

evaluate the in-vitro response with the in-silico digital twin model to generate an in-silico response; and

generate, based on the in-silico response, an output response for the change to the subject.

It is proposed to combine a multi-organ in-vitro system with a multi-organ in-silico system to provide improved modelling/prediction of multi-organ interaction(s) for a subject. By way of example, a subject-specific multi-organ in-vitro system may undergo perturbation, for example by implementing diseased or transplanted tissue, or by disturbing interactions. The response of the other organ(s) to the perturbation modelled by the in-vitro model may then be identified, and this response may then be used in the in-silico model to predict the outcome(s) of the perturbation(s).

Embodiments may therefore be of particular use for supporting clinical decision making. Exemplary usage applications may for example, relate to predicting the onset, treatment (outcome) or development of medical conditions and/or medical procedures. Embodiments may thus be of particular use in relation to organ transplantation, comorbidities e.g. cardiopulmonary interactions, and kidney failure, for example.

It is to be understood that reference to ‘in vitro’ should be taken to refer to the performance of a given procedure in a controlled environment outside of a living organism. One of the abiding weaknesses of in vitro experiments is that they fail to replicate the precise internal conditions and/or interactions within a human subject. Because of this, in vitro studies and/models may provide results that do not correspond to the circumstances occurring within a human subject.

Reference to ‘in vivo’ refers to experimentation using a whole, living subject. Animal studies and human clinical trials are two forms of in vivo research. In vivo testing is often employed over in vitro because it is better suited for observing the overall effects of an experiment on a living subject.

Use of ‘in silico’ in an expression should be taken to mean “performed on computer or via computer simulation”. Thus, the term “in silico” may be used to characterize experiments carried out entirely in a computer. There is a variety of in silico techniques, but two notable examples are: (i) Bacterial sequencing techniques; and (ii) Molecular modeling. In silico modelling is a logical extension of controlled in vitro experimentation. In silico modelling combines the advantages of both in vivo and in vitro experimentation, without subjecting itself to the ethical considerations and lack of control associated with in vivo experiments. Unlike in vitro experiments, which exist in isolation, in silico models allow a researcher to include a virtually unlimited array of parameters, which render the results more applicable to the subject as a whole.

According to proposal, an in-silico multi-organ model (i.e. a biophysical model or digital model of a biophysical organism) may be leveraged to process outputs from an in-vitro model. The in-silico multi-organ model may represent subject-specific organ interactions (quantified in the in-vitro model) so as to predict future medical responses and/or conditions of the subject.

Embodiments combine in-vitro monitoring and in-silico analysis. The in-vitro monitoring is used to determine the response of a plurality of organs to a particular stimulus, which may be an administered drug or external atmospheric or environmental conditions to which the subject may be exposed. The in-vitro model may generate parameters which cannot or are not being monitored in-vivo. For example, some subjects may not be suitable for certain monitoring functions, so the in-vitro model may be used to predict the subject response. These parameters may then supplement any actually monitored parameters of the subject when using the in-silico multi-organ model model to make predictions about the physiological condition of the subject. The physiological condition of the subject being predicted may, for example, be a condition which requires medical intervention, or else preventative action to be taken.

In an embodiment, the processor arrangement may be adapted to implement the in-silico digital twin model based on data relating to one or more interactions between the plurality of organs of the subject. In this way, the in-silico digital twin model may be configured to model subject-specific organ interactions (that may be quantified in the in-vitro system) and thus to predict future medical conditions of the human subject. By way of example, Sung et al. summarize mathematical methodologies used for the design and interpretation of multi-organ MicroPhysiological Systems (MPSs) in a paper entitled “Strategies for using mathematical modeling approaches to design and interpret multiorgan MicroPhysiological Systems (MPS)” (APL Bioeng. 3, 0215019 (2019)).

The processor arrangement may be configured to generate the output response to represent a physical condition of the subject resultant from the change to the subject. In this way, embodiments may provide useful predictions regarding the response of a subject to one or more specific changes (e.g. treatment or medical procedure).

In some embodiments, the in-vitro model may be configured to model one or more interactions between the plurality of organs of the subject. This may, for example, be based on sample cells from the subject. In this way, the in-vitro model may be configured to model interactions between the in vitro organs related to medical conditions which cannot be measured or monitored in-vivo.

In an embodiment, the processor arrangement may be further configured to generate a control instruction for a sensor or medical equipment based on the generated output response. In this way, a sensor and/or medical equipment may controlled according to results (e.g. predictions) generated by combined use of an in-vitro model and in-silico digital twin model. Dynamic and/or automate control concepts may therefore be realized by proposed embodiments.

According to examples in accordance with another aspect of the invention, there is provided a method for controlling a system for subject-specific clinical decision support comprising an in-vitro model configured to model a plurality of organs of a subject and a processor arrangement adapted to implement an in-silico digital twin model configured to model the plurality of organs of the subject. The method comprises:

providing input data to the in-vitro model, the input data being representative of a change to the subject,

evaluating the input data with the in-vitro model to generate an in-vitro response;

evaluating the in-vitro response with the in-silico digital twin model to generate an in-silico response; and

generating, based on the in-silico response, an output response for the change to the subject.

According to examples in accordance with yet another aspect of the invention, there is provided a computer program product comprising computer program code means which, when executed on a computing device having a processing system, cause the processing system to perform all of the steps of the method described above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 depicts an exemplary embodiment of a system for subject-specific clinical decision support;

FIG. 2 shows a flow diagram of a method for controlling a system for subject-specific clinical decision support; and

FIG. 3 illustrates an example of a computer within which one or more parts of an embodiment may be employed

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for modelling multi-organ interactions and responses for a subject. Such concepts may employ a multi-organ in-vitro model along with a multi-organ in-silico digital twin model. By employing output results (e.g. response data/information) from the multi-organ in-vitro model as the input(s) to the in-silico digital twin model, improved (e.g. more accurate) medical outcome predictions may be obtained. That is, output data from a subject-specific multi-organ in-vitro model may be used in an in-silico digital twin model for the subject (so as to predict medical conditions and/or responses of the subject for example).

Proposed embodiments may therefore leverage the benefits of both in-vitro models and in-silico models.

According to an exemplary embodiment, a subject-specific multi-organ in-vitro system may undergo perturbation, for example by implementing diseased or transplanted tissue, or by disturbing interactions. The response to the perturbation modelled by the in-vitro model may then be determined and used as an input to an in-silico model to predict the outcome(s) of the perturbation(s). Such embodiments may, for example, be employed to predict organ transplantation outcome(s), comorbidities, or organ failure.

By coupling the multi-organ in-vitro model results to the multi-organ in-silico model, the in-vitro results serve as an input for the in-silico model, and consequently a versatile method for determining the effect of external stimuli on the clinical disorder can be predicted and measures can be taken to prevent or minimize the clinical disorder.

Embodiments provide validated input parameters to enable accurate model predictions in the multi-organ in-silica system, with the possibility of real-time updates of the model input parameters. The inputs may also otherwise not be accessible or only intrusively accessible.

Proposed embodiments may therefore enable more accurate modeling of the state of a subject and their need for imminent medical intervention.

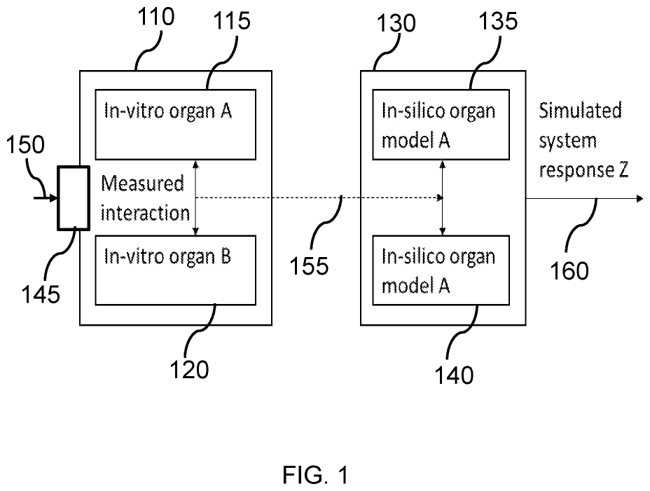

Referring now to FIG. 1, there is depicted an exemplary embodiment of a system for subject-specific clinical decision support according to an embodiment.

The system comprises an in-vitro model 110 (hosted by a laboratory) that is configured to model a plurality of organs of a subject. Specifically, the in-vitro model 110 comprises a first in-vitro model 115 of a first organ (A) of the subject, and a second in-vitro model 120 of a second organ (B) of the subject. These in-vitro models 115, 120 may, for example, be developed based on sample cells from the subject. For instance, each in-vitro model 115, 120 can be configured to model a respective tissue or organ type of the subject based on sample cells obtained from the subject.

The in-vitro models 115, 120 may each comprise input parameters relating to actual or predicted external stimuli to which the subject's organ is or may be exposed, wherein each in-vitro model 115,120 is adapted to output subject-specific parameters which are not being monitored, or cannot be monitored, in-vivo.

The in-vitro models 115, 120 of this example each comprise a microfluidic system. Such systems are for example known as lab-on-a-chip systems, and they enable tissue samples to be exposed to different external conditions and to have their responses monitored. The subject-specific parameters of the in-vitro subject model for example comprise one or more of: a tissue thickness (e.g. indicating swelling or contraction); an amount of stretching of tissue in response to an applied pressure; a tissue stiffness; and a contraction power of muscle tissue.

The in-vitro models 115, 120 output subject-specific parameters which are not being monitored directly from the subject, or cannot be monitored, in-vivo. The in-vitro models 115, 120 thus represent an important element of a biophysical model which is not accessible for “in-vivo” measurements with individual subjects, for example an in body lining tissue.

Using the first 115 and second 120 in-vitro models, the in-vitro model 110 models interactions between the first A and second B organs of the subject, This may, for example, include modelling interactions between the in-vitro organs related to specific medical conditions which cannot normally be measured or monitored in-vivo.

The system also comprises a processor arrangement 130 (hosted by a computer) that is adapted to implement an in-silico digital twin model for modelling the first A and second B organs of the subject. Specifically, the processor arrangement 130 comprises a first in-silico model 135 of the first organ A of the subject, and a second in-silico model 140 of a second organ B of the subject. The first 135 and second 140 in-silico models each comprise subject-specific data which is relevant for the response of the subject to subject-specific parameters.

The subject-specific data of the in-silico subject models 135, 140 for example comprises scan data. This may be ultrasound or magnetic resonance scan data, for example, 20 The scan data may comprise one or more of: the airway geometry; and the geometry of an organ of the subject.

Each in-silico model 135, 140 is provided with information about the external stimuli and/or time. Based on the subject-specific parameters (which provide information concerning the relationship between the tissue behavior and the stimuli), the subject-specific parameters can be taken into account and processed to predict the response of the subject to the actual or predicted external stimuli and thereby predict an onset of a physiological condition of the subject. The first 135 and second 140 in-silico model may thus be used to model subject-specific inter-organ interactions (quantified in the in-vitro system 110).

An input interface 145 of the in-vitro system 110 is configured to provide input data 150 (i.e. input parameters) to the in-vitro models 115 and 120. The input data 150 is representative of a change to the subject. The input parameters to the in-vitro system 110 for example comprise one or more of: exposure to a medicinal drug; exposure to pollutants; exposure to pressure; exposure to particular concentrations of oxygen; and exposure to vibration. These are external conditions to which the live subject may be exposed, and the in-vitro system provides a mechanism for modeling a multi-organ the response to these external stimuli.

Also, a sensor arrangement may be provided for providing sensor information as input data. That is, the input interface 145 may receive sensor information from a remote source, wherein the sensor information is provided to the in-vitro system 110 as input data. The sensor information is for sensing conditions which affect the subject, and the in-vitro models can be controlled accordingly to model the multi-organ response to those conditions.

In this way, a change or perturbation may be made to the in-vitro model which then generates an in-vitro response 155.

The generated in-vitro response 155 is provided to the processor arrangement 130 model. The first 135 and second 140 in-silico model in-silico digital twin models evaluate the in-vitro response 155 to generate an in-silico response. Responsive to this, the in-silico digital twin model implemented by the processor arrangement 130 generates, based on the in-silico response, an output response 160. The output response 160 is indicative of the subject's response to the change or perturbation. That is, the processor arrangement 130 generates the output response 160 to represent a physical condition of the subject resultant from the change to the subject (the change being represented by the input data 150).

The embodiment of FIG. 1 thus combines multi-organ in-vitro monitoring and multi-organ in-silico analysis. The in-vitro monitoring is used to determining the response of multiple organs to a particular stimuli, which may be a drug which may be administered or external atmospheric or environmental conditions to which the subject may be exposed. The in-vitro model generates parameters which cannot or are not being monitored in-vivo. These parameters supplement the actually monitored parameters of the subject when using the in-silico digital twin model to make predictions about the physiological condition of the subject. The physiological condition of the subject being predicted may, for example, comprise a condition which requires medical intervention, or else preventative action to be taken.

In some embodiments, the processor arrangement 130 be further configured to generate a control instruction for a sensor or medical equipment based on the generated output response 160. For example, such a control signal may comprise display control instructions for controlling a display screen to display information about the subject's response to the change or perturbation. In another example, the control signal may comprise control instructions for medical equipment, thus enabling the medical equipment to be automatically and/or pre-emptively controlled base on the change/perturbation.

In yet further examples, the processor arrangement 130 may be further adapted to derive treatment recommendations. Thus, the system may predict the onset of conditions which will need treatment as well as recommending early treatment interventions.

Some embodiments may therefore output information in the form of a personalized treatment advice to a physician, or feedback to a subject (e.g. instructions to adapt medication or behavior).

The system may further comprise a user input for receiving an indication of the level of the physiological condition experienced by the subject. This enables the in-silico models 135, 140 to update in response to feedback from the subject relating to the accuracy of information provided by the system.

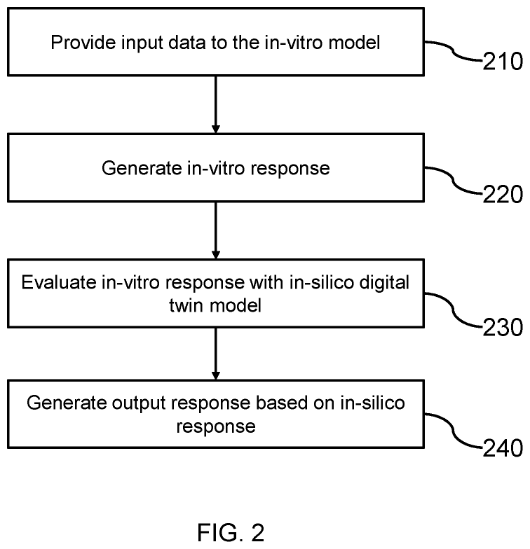

FIG. 2 depicts a flow diagram of a method for controlling a system for subject-specific clinical decision support comprising an in-vitro model configured to model a plurality of organs of a subject and a processor arrangement adapted to implement an in-silico digital twin model configured to model the plurality of organs of the subject.

The method comprises step 210 of providing input data to the in-vitro model, the input data being representative of a change to the subject.

Step 220 comprises evaluating the input data with the in-vitro model to generate an in-vitro response according to one or more interactions between the plurality of organs of the subject.

Step 230 comprises evaluating the in-vitro response with the in-silico digital twin model to generate an in-silico response.

Step 240 comprises generating, based on the in-silico response, an output response for the change to the subject. The output response may for example represent a physical condition of the subject resultant from the change to the subject.

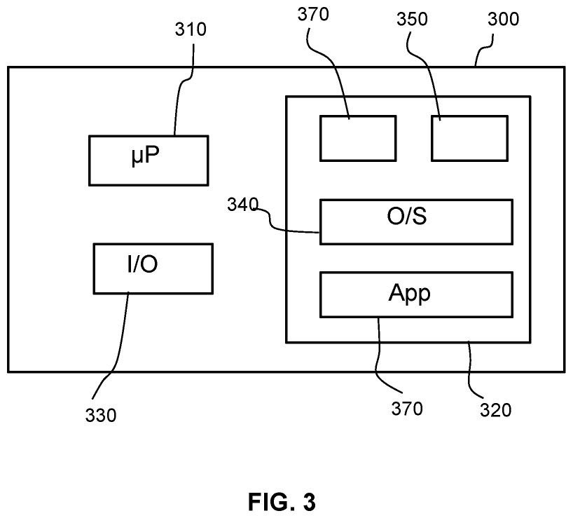

By way of further example, FIG. 3 illustrates an example of a computer 300 within which one or more parts of an embodiment may be employed. Various operations discussed above may utilize the capabilities of the computer 300. For example, the in-silico digital twin model for modelling a plurality of organs of a subject may be incorporated in any element, module, application, and/or component discussed herein. In this regard, it is to be understood that system functional blocks can run on a single computer or may be distributed over several computers and locations (e.g. connected via internet).

The computer 300 includes, but is not limited to, PCs, workstations, laptops, PDAs, palm devices, servers, storages, and the like. Generally, in terms of hardware architecture, the computer 300 may include one or more processors 310, memory 320, and one or more I/O devices 370 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 310 is a hardware device for executing software that can be stored in the memory 320. The processor 310 can be virtually any custom made or commercially available processor, a central processing unit (CPU), a digital signal processor (DSP), or an auxiliary processor among several processors associated with the computer 300, and the processor 310 may be a semiconductor based microprocessor (in the form of a microchip) or a microprocessor.

The memory 320 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 320 may incorporate electronic, magnetic, optical, and/or other types of storage media. Note that the memory 320 can have a distributed architecture, where various components are situated remote from one another, but can be accessed by the processor 310.

The software in the memory 320 may include one or more separate programs, each of which comprises an ordered listing of executable instructions for implementing logical functions. The software in the memory 320 includes a suitable operating system (O/S) 350, compiler 340, source code 330, and one or more applications 360 in accordance with exemplary embodiments. As illustrated, the application 360 comprises numerous functional components for implementing the features and operations of the exemplary embodiments. The application 360 of the computer 300 may represent various applications, computational units, logic, functional units, processes, operations, virtual entities, and/or modules in accordance with exemplary embodiments, but the application 360 is not meant to be a limitation.

The operating system 350 controls the execution of other computer programs, and provides scheduling, input-output control, file and data management, memory management, and communication control and related services. It is contemplated by the inventors that the application 360 for implementing exemplary embodiments may be applicable on all commercially available operating systems.

Application 360 may be a source program, executable program (object code), script, or any other entity comprising a set of instructions to be performed. When a source program, then the program is usually translated via a compiler (such as the compiler 340), assembler, interpreter, or the like, which may or may not be included within the memory 320, so as to operate properly in connection with the O/S 350. Furthermore, the application 360 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 370 may include input devices such as, for example but not limited to, a mouse, keyboard, scanner, microphone, camera, etc. Furthermore, the I/O devices 370 may also include output devices, for example but not limited to a printer, display, etc. Finally, the I/O devices 370 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 370 also include components for communicating over various networks, such as the Internet or intranet.

If the computer 300 is a PC, workstation, intelligent device or the like, the software in the memory 320 may further include a basic input output system (BIOS) (omitted for simplicity). The BIOS is a set of essential software routines that initialize and test hardware at startup, start the O/S 350, and support the transfer of data among the hardware devices. The BIOS is stored in some type of read-only-memory, such as ROM, PROM, EPROM, EEPROM or the like, so that the BIOS can be executed when the computer 300 is activated.

When the computer 300 is in operation, the processor 310 is configured to execute software stored within the memory 320, to communicate data to and from the memory 320, and to generally control operations of the computer 300 pursuant to the software. The application 360 and the O/S 350 are read, in whole or in part, by the processor 310, perhaps buffered within the processor 310, and then executed.

When the application 360 is implemented in software it should be noted that the application 360 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 360 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

A single processor or other unit may fulfill the functions of several items recited in the claims.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A system for subject-specific clinical decision support comprising:
an in-vitro model configured to model a plurality of organs of a subject;
a processor arrangement adapted to implement an in-silico digital twin model configured to model the plurality of organs of the subject; and
an input interface configured to provide input data to the in-vitro model, the input data being representative of a change to the subject,
wherein the in-vitro model is configured to evaluate the input data received from the input interface to generate an in-vitro response;
and wherein the processor arrangement is configured to:
evaluate the in-vitro response with the in-silico digital twin model to generate an in-silico response; and
generate, based on the in-silico response, an output response for the change to the subject.

2. The system of claim 1, wherein the processor arrangement is adapted to implement the in-silico digital twin model based on data relating to one or more interactions between the plurality of organs of the subject.

3. The system of claim 1, wherein the processor arrangement generates the output response to represent a physical condition of the subject resultant from the change to the subject.

4. The system of claim 1, wherein the in-vitro model is configured to model one or more interactions between the plurality of organs of the subject.

5. The system of claim 1, wherein the processor arrangement is further configured to generate a control instruction for a sensor or medical equipment based on the generated output response.

6. A method for controlling a system for subject-specific clinical decision support comprising an in-vitro model configured to model a plurality of organs of a subject and a processor arrangement adapted to implement an in-silico digital twin model configured to model the plurality of organs of the subject, the method comprising:
providing input data to the in-vitro model, the input data being representative of a change to the subject,
evaluating the input data with the in-vitro model to generate an in-vitro response;
evaluating the in-vitro response with the in-silico digital twin model to generate an in-silico response; and
generating, based on the in-silico response, an output response for the change to the subject.

7. The method of claim 6, further comprising controlling the processor to implement the in-silico digital twin model based on data relating to one or more interactions between the plurality of organs of the subject.

8. The method of claim 6, wherein the output response represents a physical condition of the subject resultant from the change to the subject.

9. The method of claim 6, wherein the in-vitro model is configured to model one or more interactions between the plurality of organs of the subject.

10. The method of claim 6, further comprising:
generating a control instruction for a sensor or medical equipment based on the generated output response.

11. A computer program product comprising computer program code means which, when executed on a computing device having a processing system, cause the processing system to perform all of the steps of the method according claim 6.
